(19) United States
(12) Patent Application Publication
(10) Pub. No.: US 2024/0108807 A1
(43) Pub. Date: Apr. 4, 2024

(54) SYRINGE CONNECTOR

(71) Applicant: KABUSHIKI KAISHA TOP, Tokyo (JP)

(72) Inventor: Issei Kawachi, Tokyo (JP)

(21) Appl. No.: 18/466,362

(22) Filed: Sep. 13, 2023

(30) Foreign Application Priority Data
Sep. 27, 2022 (JP) 2022-154187

(51) Int. Cl. A61M 5/31

(52) U.S. Cl. CPC A61M 5/31

(57) ABSTRACT

An object of the present invention is to provide a syringe connector having safety and a sufficient fitting strength with a syringe and easily manufacturable.

A syringe connector (30) according to the invention includes; a first member (40) including a connecting portion (41) fitted to a to-be-connected portion of a syringe (10); and a second member (60) including a connector flow path (66) through which fluid discharged from the syringe (10) flows. The first member (40) is made of resin containing a mixture. The second member (60) is made of resin containing no mixture.

### BACKGROUND OF THE INVENTION

### 1. Field of the Invention

The present invention relates to a connector connected to a distal end of a syringe.

### 2. Description of the Related Art

As described in Non-Patent Document 1, a syringe (an injector) is conventionally used for administration of medicinal solution, collection of blood, and so on in the medical field. Such a syringe is constituted by a barrel (an outer cylinder) in which medicinal solution or the like is stored, and a plunger (a pusher) to be inserted into the barrel.

A connector (a nozzle head) in conformity with a predetermined standard is attached to a distal end of the barrel. This connector is formed such that a needle base of an injection needle, a connector of a catheter, or the like is connectable to this connector. As the connector, there is a connector made of metal such as brass plated for a use purpose. The metal connector can maintain a fitting strength with the distal end of the barrel even in a case where the metal connector is kept stored for a long period of time.

### CITATION LIST

### Non-Patent Document

[Non-Patent Document 1] Japanese Industrial Standards (JIS) T3201-1979 “Glass Injector”

### SUMMARY OF THE INVENTION

However, in a case where the connector is manufactured by use of metal, the shape of the connector and the specification of the connector are restricted because of machining of metal. Further, since it takes a lot of trouble for the machining of metal, it requires a time cost and a monetary cost. As a suggestion for improvement, it is proposed to manufacture the connector by use of resin. In this regard, it is necessary for a material used to form a syringe connector to satisfy both a condition that a sufficient fitting strength with a distal end of a barrel is obtained and a condition that the material is a safe material with which no foreign material is mixed in medicinal solution or the like flowing from the barrel to the connector. However, it is difficult to manufacture a resin connector that can balance both of the above conditions. On that account, a connector employable instead of a metal connector, having safety, and easily manufacturable is demanded.

The present invention is achieved in order to solve the problem, and an object of the present invention is to provide a syringe connector having safety and a sufficient fitting strength with a syringe and easily manufacturable.

A syringe connector according to the present invention includes: a first member including a connecting portion fitted to a to-be-connected portion of a syringe; and a second member including a connector flow path through which fluid discharged from the syringe flows. The first member is made of resin containing a mixture. The second member is made of resin containing no mixture.

The syringe connector according to the present invention includes: the first member including the connecting portion fitted to the to-be-connected portion of the syringe; and the second member including the connector flow path through which fluid discharged from the syringe flows. The first member is made of resin containing a mixture, and the second member is made of resin containing no mixture. Accordingly, a material containing a mixture enhancing strength or rigidity is selected for the first member including the connecting portion to be fitted to a predetermined part of the syringe and requiring a high fitting force, and a safe material having no possibility that a raw material might be separated is selected for the second member including the connector flow path through which fluid discharged from the syringe flows. Thus, it is possible to form the first member and the second member by use of respective optimum materials. Accordingly, with the syringe connector according to the present invention, it is possible to achieve a safe syringe connector having a high fitting force.

Further, in the syringe connector according to the present invention, it is preferable that the mixture be fiber.

In the syringe connector according to the present invention, the resin forming the first member contains fiber, and the resin forming the second member contains no mixture. Accordingly, in the syringe connector according to the present invention, the first member is configured to contain fiber easily mixed into resin, so that the first member has a high fitting force with the syringe, and the second member containing no fiber is formed not to contain fiber that might be separated into medicinal solution, so that the second member has high safety.

Further, in the syringe connector according to the present invention, it is preferable that the first member and the second member be connected to each other only by fitting between respective abutment surfaces of the first member and the second member, the respective abutment surfaces making contact with each other.

In the syringe connector according to the present invention, the first member and the second member are connected to each other only by fitting between their respective abutment surfaces making contact with each other. Since other members such as an adhesive are not used, other substances are not mixed in the fluid discharged from the syringe. Accordingly, it is possible to achieve a syringe connector having higher safety.

The first member and the second member include paired fitting portions formed to be paired on the respective abutment surfaces abutting with each other, such that the paired fitting portions are fitted to each other by recess-projection fitting.

Further, in the syringe connector according to the present invention, it is preferable that the first member and the second member include paired fitting portions formed to be paired with each other on the respective abutment surfaces abutting with each other, such that the fitting portions are fitted to each other by recess-projection fitting.

The first member and the second member provided in the syringe connector according to the present invention include the paired fitting portions formed on the respective abutment surfaces abutting with each other, such that the paired fitting portions are fitted to each other by recess-projection fitting. Accordingly, with the syringe connector according to the present invention, it is possible to achieve a safe syringe connector having a high fitting force by fitting that is an easy and reliable connecting method.

Further, in the syringe connector according to the present invention, it is preferable that: the first member include a first fitting portion as one of the paired fitting portions; the second member include a second fitting portion as the other one of the paired fitting portions; the first member include a first indication portion corresponding to a rotation-direction angular position of the first fitting portion around a central axis of the first member; and the second member include a second indication portion corresponding to a rotation-direction angular position of the second fitting portion around a central axis of the second member.

The first member and the second member of the syringe connector according to the present invention include the first indication portion and the second indication portion, respectively, the first indication portion and the second indication portion corresponding to respective rotation-direction angular positions of the first fitting portion and the second fitting portion. Accordingly, the first member and the second member having fitting structures fitted to each other can be easily fitted to each other only at respective specific rotation-direction angular positions. Accordingly, it is possible to efficiently manufacture the syringe connector.

In the syringe connector according to the present invention, it is preferable that the connecting portion have an inside diameter equal to or more than 94% but equal to or less than 98% of an outside diameter of the to-be-connected portion placed at a position corresponding to the connecting portion.

In the syringe connector according to the present invention, the connecting portion has an inside diameter equal to or more than 94% but equal to or less than 98% of the outside diameter of the to-be-connected portion placed at a position corresponding to the connecting portion. Thus, the connecting portion and the to-be-connected portion are formed in a dimension relationship suitable for tight fit. Accordingly, the syringe connector according to the present invention can be pressed by fitting into the to-be-connected portion of the syringe and also has a sufficient pull-out strength and a fitting strength for a long-term storage.

In the syringe connector according to the present invention, it is preferable that the connecting portion have a plate thickness in a radial direction of the connecting portion, the plate thickness being equal to or more than 2 mm but equal to or less than 3 mm in a part other than a round part or a chamfer part provided in an end part of the connecting portion in a central axis direction of the connecting portion.

Since the plate thickness, in the radial direction, of the connecting portion of the syringe connector according to the present invention is formed within the above range, the syringe connector according to the present invention can have a sufficient pull-out strength with respect to the syringe while forming defects such as a curve or a sink mark are reduced.

In the syringe connector according to the present invention, it is preferable that: the second member include a circular plate portion; and the second indication portion be a non-circular portion formed by cutting an outer peripheral part of the circular plate portion.

Since the second indication portion provided in the syringe connector according to the present invention is a non-circular portion provided in the outer peripheral part of the circular plate portion provided in the second member, the second indication portion can be formed easily. Further, since the second indication portion is placed in the outer peripheral part of the circular plate portion, the rotation-direction position of the second member is easily grasped at the time of assembling of the connector, and further, the periphery of the second indication portion is easily gripped. Accordingly, it is possible to easily manufacture the syringe connector.

In the syringe connector according to the present invention, it is preferable that: the first member include an outer tubular portion connected to a counterpart connector, and an intermediate portion connecting the connecting portion to the outer tubular portion; the connecting portion have an inner peripheral surface placed, in a radial direction of the connecting portion, between an inner peripheral surface of the outer tubular portion and an outer peripheral surface of the outer tubular portion; and the connecting portion have an outer peripheral surface placed outwardly in the radial direction from the outer peripheral surface of the outer tubular portion.

In the first member of the syringe connector according to the present invention, the inner peripheral surface of the connecting portion is placed, in the radial direction, between the inner peripheral surface of the outer tubular portion and the outer peripheral surface of the outer tubular portion, and the outer peripheral surface of the connecting portion is placed outwardly in the radial direction from the outer peripheral surface of the outer tubular portion. Accordingly, while the fitting strength is raised by moderately restricting deformation in an outer peripheral part of the connecting portion, the connecting portion is held by the outer tubular portion to prevent an excessive stress from occurring locally, thereby making it possible to enhance the fitting strength stably.

It is preferable that: the syringe connector according to the present invention include a sealing member placed to be sandwiched between the syringe and the syringe connector in a central axis direction of the syringe connector, the sealing member being more easily deformable than the syringe connector; and the syringe connector have a groove continuous in a circumferential direction of the syringe connector on a surface making contact with the sealing member.

The syringe connector according to the present invention includes the sealing member between the syringe and the syringe connector, the sealing member being more easily deformable than the syringe connector, and the syringe connector has the groove continuous in the circumferential direction on the surface making contact with the sealing member. With the above configuration, the syringe connector according to the present invention causes the sealing member to swell in the groove, and due to the physical shape of a corner part of the groove, sealing performance improves in comparison with a case where no groove is formed.

It is preferable that: the syringe connector according to the present invention include a connecting portion having an inner peripheral surface formed in a truncated cone shape or a cylindrical shape; and the connecting portion be outwardly engaged by tight fit with a to-be-connected portion provided in a glass barrel of the syringe and having an outer peripheral surface formed in a truncated cone shape or a cylindrical shape.

The syringe connector according to the present invention includes the connecting portion having an inner peripheral surface formed in a truncated cone shape or a cylindrical shape, and the connecting portion is outwardly engaged by tight fit with the to-be-connected portion provided in the glass barrel of the syringe and having an outer peripheral surface formed in a truncated cone shape or a cylindrical shape. This accordingly facilitates insertion into the connector and increases an adhesion property with the connector, thereby making it possible to enhance the fitting strength.

### BRIEF DESCRIPTION OF THE DRAWINGS

FIG. 1 is a perspective view illustrating a state where a connector according to an embodiment of the present invention is connected to a syringe,

FIG. 2 is a side view of the connector and the syringe in FIG. 1,

FIG. 3 is a sectional view of a section parallel to a central axis passing through the connector and the syringe in FIG. 2,

FIG. 4 is a right-side view of a first member in FIG. 1,

FIG. 5 is a right-side view of a second member in FIG. 1, and

FIG. 6 is a left-side view of the second member in FIG. 1.

### DESCRIPTION OF THE PREFERRED EMBODIMENTS

### Embodiment

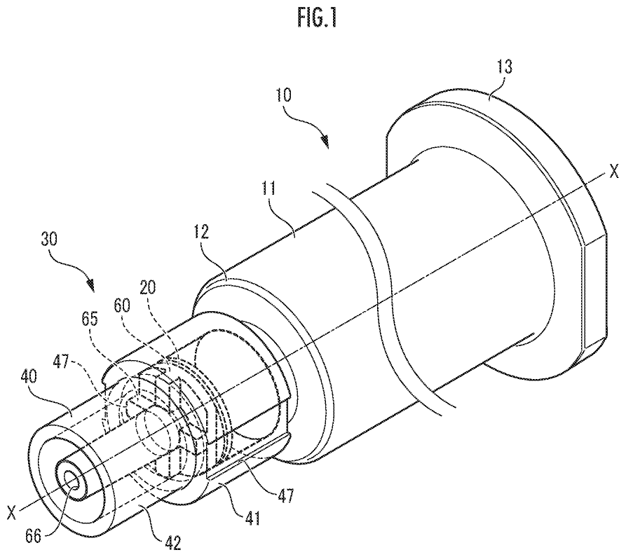

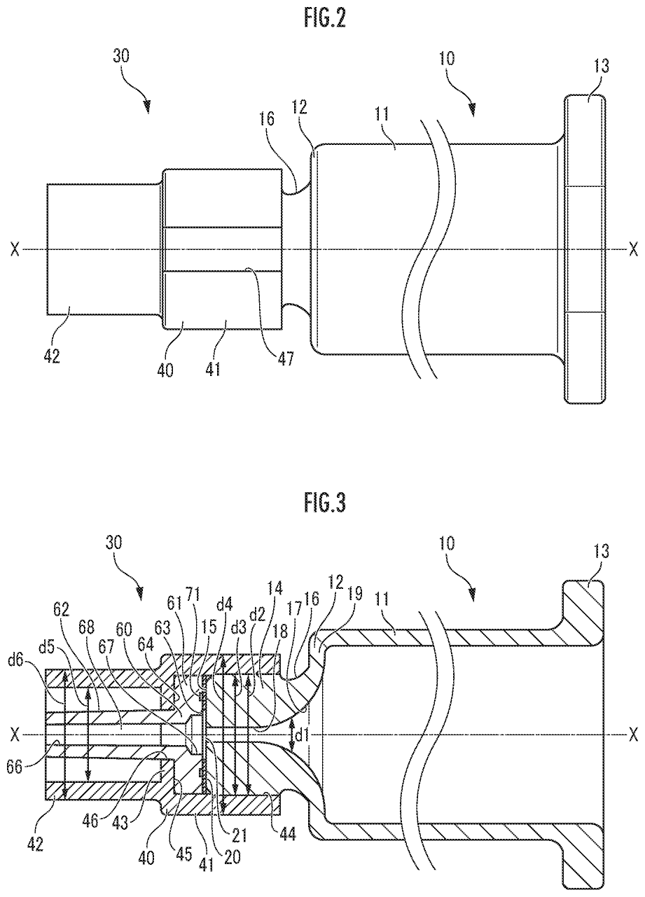

With reference to FIGS. 1 to 3, a connector 30 according to an embodiment of the present invention will be described. FIG. 1 is a perspective view illustrating a state where the connector 30 is attached to a syringe 10. FIG. 2 is a front view of the connector 30 and the syringe 10 in FIG. 1. FIG. 3 is a sectional view along a plane including a central axis X in FIG. 2. The connector 30 is configured to be attached to a syringe corresponding to the ISO80369-6 standard.

The syringe 10 is used for administration of medicinal solution, collection of blood, or the like, and as publicly known, the syringe 10 is constituted by a barrel 11 and a plunger (not illustrated). The barrel 11 is formed into a generally cylindrical shape having an internal space in which fluid such as medicinal solution, saline, or air is stored. The barrel 11 includes a distal end portion 12 in an end part, on a connector side, of the barrel 11, and a flange portion 13 in an end part on an opposite side from the distal end portion 12. The barrel 11 is made of glass and is made of borosilicate glass, for example.

The distal end portion 12 includes a to-be-connected portion 14 formed to project from a barrel end surface 19 on the distal end portion 12 side of the barrel 11 formed into a generally cylindrical shape. The to-be-connected portion 14 is a part formed with an outside diameter d2, connected to the barrel end surface 19 via a neck portion 16, and having an outer peripheral surface with a truncated cone shape or a cylindrical shape. A to-be-connected portion distal surface 15 is formed on a distal surface on the connector side of the to-be-connected portion 14. The to-be-connected portion 14 and the neck portion 16 are formed integrally as part of the barrel 11. Inside the to-be-connected portion 14, a syringe flow path 18 communicating with an internal space of the barrel 11 and penetrating along a central axis X of the to-be-connected portion 14 from the barrel end surface 19 to the to-be-connected portion distal surface 15 is provided. An inside diameter d1 on the barrel end surface 19 side of the syringe flow path 18 is a maximum inside diameter part of the inside diameter d1 of the syringe flow path 18, and the inside diameter d1 of the syringe flow path 18 is formed to have a generally truncated cone shape gradually reduced in diameter toward the to-be-connected portion distal surface 15. On the to-be-connected portion distal surface 15 side of the syringe flow path 18, the inside diameter d1 is formed to be uniform, and the syringe flow path 18 is opened on the to-be-connected portion distal surface 15.

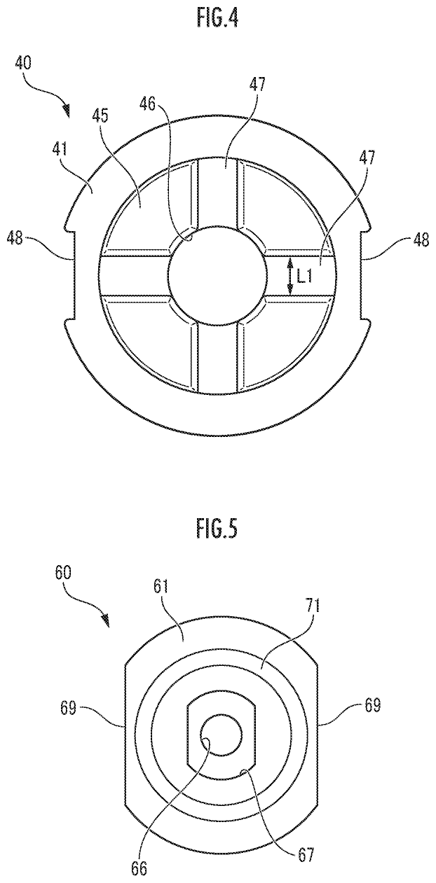

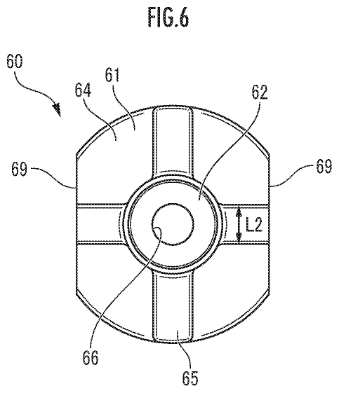

Referring now to FIGS. 3 to 6, the connector 30 will be described. The connector 30 includes a first member 40 and a second member 60 fitted and stored in the first member 40. FIG. 4 is a right-side view of the first member 40 viewed from the right side in FIG. 3, that is, viewed from the syringe 10 side. FIG. 5 is a right-side view of the second member 60 viewed from the right side in FIG. 3, that is, viewed from the syringe 10 side. FIG. 6 is a left-side view of the second member 60 viewed from the left side in FIG. 3, that is, viewed from the distal end side of the connector 30.

The first member 40 includes a connecting portion 41 having a cylindrical shape and configured to be fitted to the outer surface of the to-be-connected portion 14, an outer tubular portion 42 connected to the connecting portion 41 coaxially along the central axis X, and an intermediate portion 43 connecting the connecting portion 41 to the outer tubular portion 42. The connecting portion 41 is a part fitted to the to-be-connected portion 14 of the syringe 10. The inner peripheral surface of the connecting portion 41 is formed into a truncated cone shape or a cylindrical shape with an inside diameter d3 such that the inner peripheral surface of the connecting portion 41 is outwardly engaged with the to-be-connected portion 14.

The outer tubular portion 42 is a cylindrical portion formed of a cylindrical surface parallel to the central axis X, and a syringe side edge of the outer tubular portion 42 is connected to a distal end side surface of the intermediate portion 43. The outer tubular portion 42 may include a screw ridge portion on the inner surface of the outer tubular portion 42, the screw ridge portion being threadedly engageable with a threaded portion provided in a female connector or the like (not illustrated) to which the connector 30 is connected. In that case, the connector 30 can be also used for a counterpart connector including a collar having a threaded groove.

The intermediate portion 43 is fixed to close an end part of a distal end side of the connecting portion 41 having a cylindrical shape. The intermediate portion 43 includes an intermediate-portion first end surface 45 as a surface on the syringe 10 side, and an intermediate-portion central hole 46 formed in the center of the intermediate portion 43. The intermediate-portion central hole 46 is a hole through which an inner tubular portion 62 of the second member 60 (described later) penetrates and is formed to have an inside diameter with generally the same dimension as the outside diameter of a base end part of the inner tubular portion 62 connected to the intermediate portion 43. The intermediate-portion central hole 46 may be formed with an inside diameter slightly smaller than the outside diameter of the base end part of the inner tubular portion 62 of the second member 60, that is, an edge of the inner tubular portion 62 on the intermediate-portion first end surface 45 side, such that respective curved surfaces of the intermediate-portion central hole 46 and the inner tubular portion 62 are brought into surface contact with each other and fitted to each other by tight fit. Alternatively, a recessed portion may be provided on either one of the inner peripheral surface of the intermediate-portion central hole 46 and the outer peripheral surface of the inner tubular portion 62 with which the inner peripheral surface makes contact, and a projection portion may be provided on the other one of them, such that the recessed portion and the projection portion are fitted to each other by recess-projection fitting.

The inside diameter of the inner peripheral surface of the connecting portion 41 is formed to have generally the same dimension as the outside diameter of the outer peripheral surface of a circular plate portion 61 facing the inner peripheral surface of the connecting portion 41. The inside diameter of the inner peripheral surface of the connecting portion 41 may be formed to be slightly smaller than the outside diameter of the outer peripheral surface of the circular plate portion 61, such that respective curved surfaces of the inner peripheral surface of the connecting portion 41 and the outer peripheral surface of the circular plate portion 61 are brought into surface contact with each other and fitted to each other by tight fit. Alternatively, a recessed portion may be provided on either one of the inner peripheral surface of the connecting portion 41 and the outer peripheral surface of the circular plate portion 61 with which the inner peripheral surface makes contact, and a projection portion may be provided on the other one of them, such that the recessed portion and the projection portion are fitted to each other by recess-projection fitting.

The first member 40 and the second member 60 are fixed to each other by a fixation method other than fitting. The fixation method other than fitting is welding, for example. Respective contact parts of the first member 40 and the second member 60, e.g., the inner peripheral surface of the connecting portion 41 and the outer peripheral surface of the circular plate portion 61, or the inner peripheral surface of the intermediate-portion central hole 46 and the outer peripheral surface of the inner tubular portion 62, may be welded to each other. A welding method may be ultrasonic welding, laser welding, and the like, for example. Alternatively, bayonet fitting may be employed such that a recessed portion and a projection portion are formed in the first member 40 and the second member 60 in the rotation direction of the first member 40 and the second member 60. Alternatively, snap fitting may be employed such that a recessed portion and a projection portion are formed in the first member 40 and the second member 60 in the central-axis-X direction.

As illustrated in FIG. 4, a first fitting portion 47 as one of paired fitting portions is formed on the intermediate-portion first end surface 45. The first fitting portion 47 is a part to be fitted to a corresponding part of the second member 60 to prevent rotation. The first fitting portion 47 is one or more recessed portions, grooves or the like formed with a uniform groove width L1 in the radial direction of the first member 40 from the intermediate-portion central hole 46 to an outer peripheral end of the intermediate-portion first end surface 45. Four first fitting portions 47 are formed at an interval of 90° in the circumferential direction of the first member 40 around the intermediate-portion central hole 46 such that the four first fitting portions 47 each have the uniform groove width L1. The number of the first fitting portions 47 may be other than four, and one first fitting portion 47 may be formed in the circumferential direction of the intermediate-portion first end surface 45, or two to six fitting portions 47 may be formed at the same angle interval in the circumferential direction around the intermediate-portion central hole 46.

On the outer peripheral surface of the connecting portion 41, a first indication portion 48 corresponding to a rotation-direction angular position of the first fitting portion 47 around the central axis X of the first member 40 is provided. The first indication portion 48 is a part for positioning respective rotation-direction angular positions of the first member 40 and the second member 60 at the time of assembling of the first member 40 and the second member 60. The first indication portion 48 is a U-shaped groove formed by cutting part of the outer periphery of the connecting portion 41 from the outer periphery of the connecting portion 41 toward the center of the first member 40. The first indication portion 48 includes a straight line parallel to a straight-line making contact with the outer periphery of the connecting portion 41, and straight lines positioned in both ends of the above straight line and perpendicular to the above straight line. As illustrated in FIG. 2, the first indication portion 48 is provided over the whole length of the connecting portion 41 in the central-axis-X direction of the connecting portion 41. Two first indication portions 48 are formed at an interval of 180° on the outer periphery of the circular plate portion 61. The first indication portions 48 are provided at respective rotation-direction angular positions that are the same as their corresponding first fitting portions 47 in the rotation direction of the connecting portion 41. The first indication portions 48 may be provided at respective rotation-direction angular positions different from those of the first fitting portions 47.

The inside diameter d3 of the connecting portion 41 is formed to fall within a range of 94% to 98% of the outside diameter d2 of the to-be-connected portion 14, and the connecting portion 41 and the to-be-connected portion 14 are fitted to each other by tight fit. When the inside diameter d3 of the connecting portion 41 is less than the range, it is difficult to press the connecting portion 41 into the to-be-connected portion 14 by fitting. Further, when the inside diameter d3 of the connecting portion 41 exceeds the range, the force to fasten the connecting portion 41 decreases. As a result, it is difficult to obtain a sufficient pull-out strength of the connecting portion 41 from the to-be-connected portion 14. Further, it might be difficult to maintain a fitting strength requested by creep deformation or the like in long-term storage (e.g., a fitting strength that can tolerate a pull-out load of 78 N, defined in JIS T3201-1979, for five minutes).

The following describes the relationship between the connecting portion 41 and the outer tubular portion 42 that form the first member 40, in terms of radial dimension. The inside diameter d3 of the connecting portion 41 is larger than an inside diameter d5 of the outer tubular portion 42 but is smaller than an outside diameter d6 of the outer tubular portion 42. Further, an outside diameter d4 of the connecting portion 41 is larger than the outside diameter d6 of the outer tubular portion 42. In other words, in a direction perpendicular to the central-axis-X direction of the connector 30, namely, in the radial direction, the inner peripheral surface of the connecting portion 41 is placed between the inner peripheral surface of the outer tubular portion 42 and the outer peripheral surface of the outer tubular portion 42. Further, the outer peripheral surface of the connecting portion 41 is placed outwardly from the outer peripheral surface of the outer tubular portion 42. Hereby, while the fitting strength is raised by moderately restricting deformation in an outer peripheral part of the connecting portion 41, the connecting portion 41 is held by the outer tubular portion 42 to prevent an excessive stress from occurring locally, so that the fitting strength improves stably.

The plate thickness of the connecting portion 41 is formed in a dimension equal to or more than 2 mm but equal to or less than 3 mm in parts other than a round part or a chamfer part provided in an end of the connecting portion 41 in the central-axis-X direction. As the plate thickness of the connecting portion 41 is made thicker, the strength or the rigidity of the connecting portion 41 improves. However, as the plate thickness is made thicker than the above, the possibility to cause a forming defect such as a curve or a sink mark increases. When the connecting portion 41 is formed to have the above plate thickness, the connector 30 has a pull-out strength of 300 N or more to the syringe 10 while forming defects are reduced.

The first member 40 is made of a resin containing a mixture. The resin containing a mixture may be a mixture of a plurality of resins. The first member 40 may be made of a resin containing a mixture other than resins. Since the resin forming the first member 40 contains such a mixture, the strength or the rigidity of the first member 40 improves. The resin forming the first member 40 is preferably polyamide resin (PA). The polyamide resin is chemically and biologically safe, and in the field of medical equipment, the polyamide resin is mainly used as a raw material for surgical sutures, catheters, or the like, and thus, the polyamide resin is successfully used in the medical field. Further, the polyamide resin has a high creep resistance and has such a characteristic that its fitting strength is hard to decrease even when the connector 30 is stored for a long period of time in a state where the connector 30 is fitted to the barrel 11. The polyamide resin (PA) includes aliphatic polyamide and aromatic polyamide, and either of them is usable. The aliphatic polyamide resin includes, for example, nylon 66, nylon 6, and the like. The aromatic polyamide includes para-aramid and meta-aramid. Any mixture is usable provided that the mixture improves the strength or the rigidity of the first member 40. The mixture is, for example, fiber. The fiber includes glass fiber, aramid fiber (Kevlar fiber), carbon fiber, ZYLON fiber, polyethylene fiber (Dyneema), boron fiber, and the like. Further, the mixture may be also a carbon tube including a carbon nanotube.

The second member 60 is a member forming the connector flow path 66 through which medicinal solution or the like discharged from the syringe 10 flows. The second member 60 includes a circular plate portion 61 and an inner tubular portion 62 connected to the circular plate portion 61. The inner tubular portion 62 is connected to a circular-plate-portion second end surface 64 of the circular plate portion 61 coaxially with the circular plate portion 61 along the central axis X, such that the inner tubular portion 62 and the circular plate portion 61 are formed integrally. The second member 60 includes a connector flow path 66 formed as a through-hole provided over both the circular plate portion 61 and the inner tubular portion 62. The connector flow path 66 is a generally cylindrical space through which fluid such as the medicinal solution discharged from the syringe flow path 18 flows. The connector flow path 66 is formed to have an extremely small diameter, so that residues such as the medicinal solution remaining in the connector flow path 66 without being pushed out are reduced in comparison with a conventional technology.

The circular plate portion 61 is a disk-shaped member formed to have an outside diameter slightly smaller than the inside diameter d3 of the first member 40 and includes a circular-plate-portion first end surface 63, and the circular-plate-portion second end surface 64. A central part of the circular-plate-portion first end surface 63 is provided with an introduction portion 67 as part of the connector flow path 66, and the medicinal solution or the like discharged from the syringe flow path 18 directly flows into the introduction portion 67.

As illustrated in FIG. 5, the introduction portion 67 is configured such that a cross section of the introduction portion 67 with respect to the central-axis-X direction of the connector 30 has a shape obtained by cutting a circular shape by two straight lines generally parallel to each other. Further, the cross-sectional shape of a part of the connector flow path 66, closer to the distal end side than the introduction portion 67, with respect to the central-axis-X direction of the connector 30 is formed into a generally circular shape. The minimum inside dimension of the introduction portion 67 is formed to be larger than the maximum inside dimension of a generally circular shape forming the cross section of the part of the connector flow path 66, closer to the distal end side than the introduction portion 67. That is, the inner peripheral surface of the connector flow path 66 is formed by two stages in the central-axis-X direction. The minimum inside dimension of the introduction portion 67 is larger than the inside diameter d1 of the syringe flow path 18 on the to-be-connected portion distal surface 15 of the syringe 10 and is about two times to about three times of the inside diameter d1.

A second fitting portion 65 as the other one of the paired fitting portions is formed on the circular-plate-portion second end surface 64 of the circular plate portion 61. The second fitting portion 65 is a part to be fitted to the first fitting portion 47 formed on the intermediate-portion first end surface 45 of the first member 40 to prevent rotation. The second fitting portion 65 is a projecting portion or a protrusion formed to have a uniform width L2 in the radial direction on the intermediate-portion first end surface 45 from the inner tubular portion 62 to an outer peripheral end of the circular plate portion 61. The second fitting portion 65 is connected to the surface of a base end part of the inner tubular portion 62 such that the second fitting portion 65 is formed integrally with the surface of the base end part of the inner tubular portion 62. On that account, the second fitting portion 65 has an effect to improve the bending rigidity of the inner tubular portion 62 and to prevent the inner tubular portion 62 from bending or breaking. Four second fitting portions 65 are formed at an interval of 90° in the circumferential direction around the base end part of the inner tubular portion 62 such that the four second fitting portions 65 each have the uniform width L2. The number of the second fitting portions 65 may be other than four, provided that the second fitting portions 65 can be fitted to the first fitting portions 47. One second fitting portion 65 may be formed in the circumferential direction of the intermediate-portion first end surface 45, or two to six second fitting portions 65 may be formed at the same angle interval in the circumferential direction around the intermediate-portion central hole 46.

A sealing member 20 made of a material more easily deformable than the syringe connector 39 is placed between the circular-plate-portion first end surface 63 and the to-be-connected portion distal surface 15 of the syringe 10 such that the sealing member 20 is sandwiched between the circular-plate-portion first end surface 63 and the to-be-connected portion distal surface 15 of the syringe 10 in the central-axis-X direction. The sealing member 20 prevents the occurrence of leakage of the medicinal solution or the like. The sealing member 20 has a circular shape and has a sealing-member central hole 21 formed in a central part of the sealing member 20. The sealing member 20 is made of a thin elastic member, e.g., silicone rubber. An annular groove 71 continuous in the circumferential direction with a predetermined radius is formed on the circular-plate-portion first end surface 63. The annular groove 71 allows a part of the sealing member 20 to swell, the part facing the annular groove 71, and due to a physical shape of a corner part of the annular groove 71, formed on the circular-plate-portion first end surface 63, sealing performance improves in comparison with a case where no annular groove 71 is formed.

An outer peripheral portion of the circular plate portion 61 includes a second indication portion 69 corresponding to a rotation-direction angular position of the second fitting portion 65 around the central axis of the second member 60. The second indication portion 69 is a part for positioning respective rotation-direction angular positions of the first member 40 and the second member 60 at the time of assembling the first member 40 and the second member 60. The second indication portion 69 is a so-called D-cut shaped part obtained by cutting an arc part of the outer peripheral surface of the circular plate portion 61 by a straight line perpendicular to the radial direction, and two second indication portions 69 are formed at an interval of 180° on the outer periphery of the circular plate portion 61. In other words, the second indication portion is a non-circular portion formed by cutting an outer peripheral part of the circular plate portion 61. Each of the second indication portions 69 is provided at the same rotation-direction angular position as its corresponding second fitting portion 65 in the rotation direction of the circular plate portion 61. The second indication portions 69 may be provided at respective rotation-direction angular positions different from those of the second fitting portions 65. Accordingly, since the second indication portions 69 are non-circular portions provided in the outer peripheral part of the circular plate portion 61 provided in the second member 60, the second indication portions 69 can be formed easily. Further, since the second indication portions 69 are placed in the outer peripheral part of the circular plate portion 61, the rotation-direction angular position of the second member 60 is easily grasped at the time of assembling of the connector 30, and the periphery of the second member 60 is easily gripped.

Any resin is usable for the second member 60, provided that the resin is a chemically and biologically safe resin. The second member 60 is made of polyamide resin (PA), for example. The polyamide resin (PA) is chemically and biologically safe, and in the field of medical equipment, the polyamide resin is mainly used as a raw material for surgical sutures, catheters, or the like, and thus, the polyamide resin is successfully used in the medical field. The polyamide resin (PA) includes aliphatic polyamide and aromatic polyamide, and either of them is usable. The aliphatic polyamide resin includes, for example, nylon 66, nylon 6, and the like. The aromatic polyamide includes para-aramid and meta-aramid. Further, since the second member 60 is a member forming the connector flow path 66 through which the medicinal solution or the like flows, the second member 60 does not contain a mixture that may be separated in the medicinal solution, e.g., fiber.

The sealing-member central hole 21 and the connector flow path 66 form a flow path space 68 through which the medicinal solution or the like discharged from the syringe flow path 18 flows. The materials for forming the second member 60 and the sealing member 20 making contact with the flow path space 68 do not contain a material, e.g., grass fiber, that might partially fall off in the medicinal solution. Accordingly, the medicinal solution or the like is injected into the body of a patient without containing foreign matter.

Since the first member 40 and the second member 60 are manufactured by resin-molding, the connector 30 can be colored with a desired color at lower cost than a metal connector. In the case of a metal connector, it is necessary to color the metal connecter by providing a coating step after the shape of the connector is formed. In the meantime, in the case of the connector 30 of the present invention, manufactured by resin-molding, the first member 40 and the second member 60 can be colored with a color defined by the ISO standard, or the like by mixing pigment as a molding material. On that account, the connector 30 can be formed as the connector 30 colored with a desired color at lower cost than the metal connector.

[Assembling Method of Connector 30]

The following describes an assembling method of the connector 30. The connector 30 is assembled by putting the inner tubular portion 62 of the second member into the intermediate-portion central hole 46 of the first member 40. At the time of assembling, by use of the first indication portion 48 and the second indication portion 69 with a jig or an automated assembly machine, the first member 40 and the second member 60 are held in a state where their rotation-direction angular positions around the central axis X are aligned with each other. While this state is maintained, the inner tubular portion 62 is put through the intermediate-portion central hole 46, such that the second fitting portion 65 of the second member 60 is fitted to the first fitting portion 47 of the first member 40, and hereby, the assembling of the connector 30 is finished.

### MODIFICATIONS

The above embodiment can be configured by replacing part of the configuration of the above embodiment with the following configurations.

The first fitting portion 47 and the second fitting portion 65 may have different shapes, provided that the first fitting portion 47 and the second fitting portion 65 are surely fitted to each other. For example, the first fitting portion 47 and the second fitting portion 65 may be formed to project and to be recessed, respectively, in a reverse manner to the above. Alternatively, instead of the recessed portion and the projection portion extending in the radial direction, the first fitting portion 47 and the second fitting portion 65 may respectively include a recessed portion and a projection portion formed to extend in the circumferential direction at positions corresponding to each other. Alternatively, the recessed portion and the projection portion may be a recessed portion and a projection portion corresponding to respective given shapes formed in corresponding parts of the intermediate-portion first end surface 45 and the circular-plate-portion first end surface 63.

Further, the first indication portion 48 and the second indication portion 69 may have other shapes, provided that the first indication portion 48 and the second indication portion 69 clearly indicate respective rotation-direction angular positions of the first member 40 and the second member 60. For example, the first indication portion 48 and the second indication portion 69 may be respective arcuate grooves. Alternatively, the first indication portion 48 and the second indication portion 69 may be omitted. In a case where the first indication portion 48 and the second indication portion 69 are omitted, the first member 40 and the second member 60 have respective outer shapes illustrated by broken lines in FIGS. 4, 5.

With the present invention, it is possible to provide a syringe connector having safety and a sufficient fitting strength with a syringe and easily manufacturable.

### DESCRIPTION OF REFERENCE NUMERALS

10: syringe, 40: first member, 47: first fitting portion (fitting portion), 48: first indication portion, 60: second member, 61: circular plate portion, 65: second fitting portion (fitting portion), 68: flow path space, 69: second indication portion

What is claimed is:

1. A syringe connector comprising:
a first member including a connecting portion fitted to a to-be-connected portion of a syringe; and
a second member including a connector flow path through which fluid discharged from the syringe flows, wherein:
the first member is made of resin containing a mixture; and
the second member is made of resin containing no mixture.

2. The syringe connector according to claim 1, wherein the mixture is fiber.

3. The syringe connector according to claim 2, wherein the first member and the second member are fixed to each other only by fitting between respective abutment surfaces of the first member and the second member, the respective abutment surfaces abutting with each other.

4. The syringe connector according to claim 3, wherein
the first member and the second member include paired fitting portions formed to be paired with each other on the respective abutment surfaces abutting with each other, such that the fitting portions are fitted to each other by recess-projection fitting.

5. The syringe connector according to claim 4, wherein:
the first member includes a first fitting portion as one of the paired fitting portions;
the second member includes a second fitting portion as the other one of the paired fitting portions;
the first member includes a first indication portion corresponding to a rotation-direction angular position of the first fitting portion around a central axis of the first member; and
the second member includes a second indication portion corresponding to a rotation-direction angular position of the second fitting portion around a central axis of the second member.

6. The syringe connector according to claim 1, wherein
the connecting portion has an inside diameter equal to or more than 94% and equal to or less than 98% of an outside diameter of the to-be-connected portion placed at a position corresponding to the connecting portion.

7. The syringe connector according to claim 1, wherein
the connecting portion has a plate thickness in a radial direction of the connecting portion, the plate thickness being equal to or more than 2 mm and equal to or less than 3 mm in a part other than a round part or a chamfer part provided in an end part of the connecting portion in a central axis direction of the connecting portion.

8. The syringe connector according to claim 5, wherein:
the second member includes a circular plate portion, and
the second indication portion is a non-circular portion formed by cutting an outer peripheral part of the circular plate portion.

9. The syringe connector according to claim 1, wherein:
the first member includes an outer tubular portion connected to a counterpart connector, and an intermediate portion provided between the connecting portion and the outer tubular portion which connects the connecting portion to the outer tubular portion;
the connecting portion has an inner peripheral surface placed, in a radial direction of the connecting portion, between an inner peripheral surface of the outer tubular portion and an outer peripheral surface of the outer tubular portion; and
the connecting portion has an outer peripheral surface placed outwardly in the radial direction from the outer peripheral surface of the outer tubular portion.

10. The syringe connector according to claim 1, further comprising:
a sealing member placed to be sandwiched between the syringe and the syringe connector in a central axis direction of the syringe connector, the sealing member being more easily deformable than the syringe connector, wherein:
the syringe connector has a groove continuous in a circumferential direction of the syringe connector on a surface in contact with the sealing member.

11. The syringe connector according to claim 1, wherein:
the connecting portion has an inner peripheral surface formed in a truncated cone shape or a cylindrical shape; and
the connecting portion is outwardly engaged by tight fit with the to-be-connected portion provided in a glass barrel of the syringe and having an outer peripheral surface formed in a truncated cone shape or a cylindrical shape.
